(19) United States
(12) Patent Application Publication
(10) Pub. No.: US 2022/0409631 A1
(43) Pub. Date: Dec. 29, 2022

(54) METHODS OF INDUCING OR ENHANCING FARNESOID X RECEPTOR (FXR)-MEDIATED TRANSCRIPTIONAL RESPONSE

(71) Applicant: YALE UNIVERSITY, New Haven, CT (US)

(72) Inventors: YungChi Cheng, Woodbridge, CT (US); Wing Lam, New Haven, CT (US)

(21) Appl. No.: 17/776,922

(22) PCT Filed: Nov. 19, 2020
(86) PCT No.: PCT/US2020/061257

§ 371 Date: May 13, 2022

Related U.S. Application Data
(60) Provisional application No. 62/937,964, filed on Nov. 20, 2019.

(51) Int. Cl. A61K 31/575; A61K 36/07

(52) U.S. Cl. CPC A61K 31/575; A61K 36/07

(57) ABSTRACT

The disclosure provides a method of treating disease or disorder in a subject in need thereof by administering a therapeutically effective amount of an extract comprising at least one selective Farnesoid X receptor (FXR) agonist obtained from Antrodia cinnamomea (Antrodia camphorate) to the subject. The disease or the disorder includes liver disease, obesity, diabetes, diarrhea, abdominal pain, hypertension, itchy skin, liver cancer, hepatitis, biliary cholangitis, nonalcoholic steatohepatitis, primary sclerosing cholangitis, inflammation, and fibrosis.

### CROSS-REFERENCE TO RELATED APPLICATION

This application claims the benefit of priority to U.S. Provisional Patent Application Ser. No. 62/937,964 entitled “METHODS OF INDUCING OR ENHANCING FARNESOID X RECEPTOR (FXR)-MEDIATED TRANSCRIPTIONAL RESPONSE,” filed Nov. 20, 2019, the disclosure of which is incorporated herein by reference in its entirety.

### BACKGROUND OF THE INVENTION

Farnesoid X receptor (FXR) belongs to the family of nuclear receptors. FXR is classified as a nuclear bile acid receptor because its endogenous ligands are bile acids such as, for example, chenodeoxycholic acid (CDCA) and cholic acid. FXR is highly expressed in liver, intestine, kidney, and adrenal glands. In its inactive form, FXR forms a heterodimer with Retinoid X receptor (RXR), binds to DNA (having consensus sequence AGGTCANTGACCT (SEQ ID NO: 1), and complexes with a co-repressor. When FXR binds to its ligand, the co-activator replaces the co-repressor and promotes the activity of FXR-RXR dimer, thereby directly or indirectly turning on the genes related to bile acid synthesis and transport, lipid metabolism, and glucose homeostasis.

Agonists of FXR are considered to have therapeutic value for treating liver diseases, obesity, and diabetes. Therefore there is a need in the art to develop the molecules that can act as FXR agonist. The present invention fulfills this need.

### BRIEF SUMMARY OF THE INVENTION

Provided herein are methods of treating diseases and disorders with a compound of formula (I), formula (I-A), formula (II), formula (II-A), or mixtures thereof. The compounds are useful for treating or ameliorating a variety of diseases and disorders, including liver disease, obesity, diabetes, diarrhea, abdominal pain, hypertension, itchy skin, liver cancer, hepatitis, biliary cholangitis, nonalcoholic steatohepatitis, primary sclerosing cholangitis, inflammation, and fibrosis.

### BRIEF DESCRIPTION OF THE DRAWINGS

The following detailed description of specific embodiments of the invention will be better understood when read in conjunction with the appended drawings. For the purpose of illustrating the invention, exemplary embodiments are shown in the drawings. It should be understood, however, that the invention is not limited to the precise arrangements and instrumentalities of the embodiments shown in the drawings.

FIGS. 1A-1D show effects of compounds (I) and (II) on FXR-mediated transcriptional response without CDCA (FIG. 1A) and with CDCA (FIG. 1B) or LXR mediated transcriptional response without GW3965 (FIG. 1C) and with GW3965 (FIG. 1D).

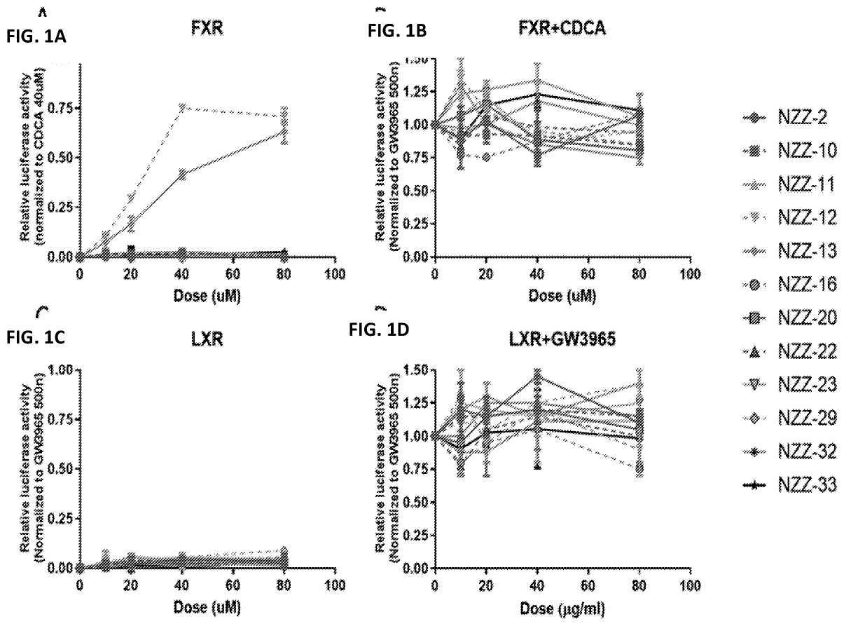

FIGS. 2A-2D show effects of CDCA, compound (I), and compound (II) on the expression of FXR downstream target genes—CYP7A1 (FIG. 2A), PPARa (FIG. 2B), BACS (FIG. 2C), and KLF11 (FIG. 2D) of HepG2 cells.

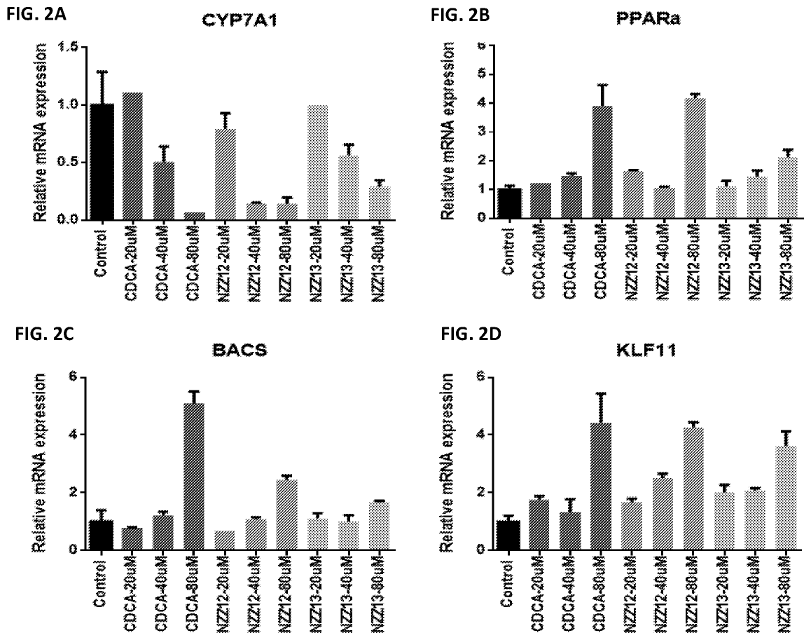

FIG. 3 shows structures of triterpenoids obtained from purifying an extract of the fruit body of Antrodia camphorate.

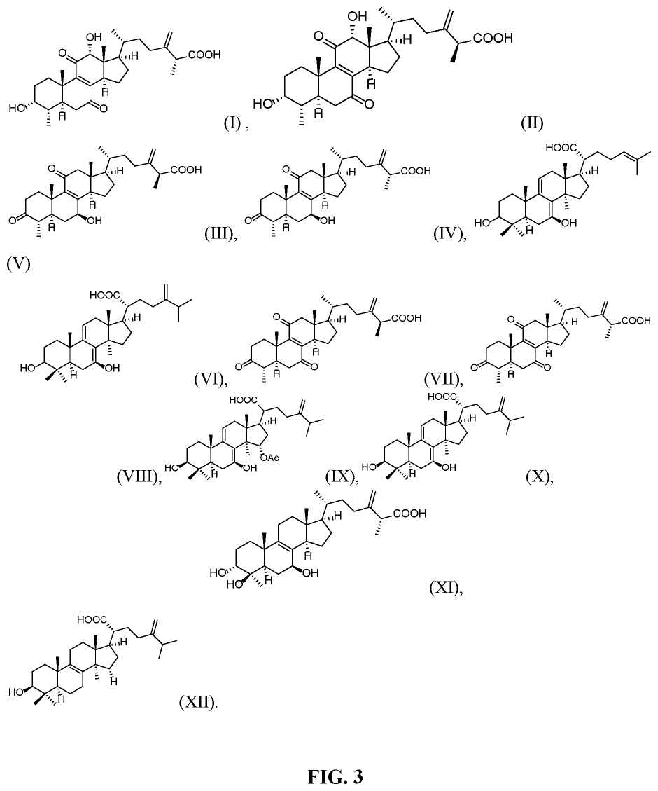

FIG. 4 is a table showing molecular formulae (MF), molecular weights (MW), and names of triterpenoids obtained from purifying an extract of the fruit of Antrodia camphorate.

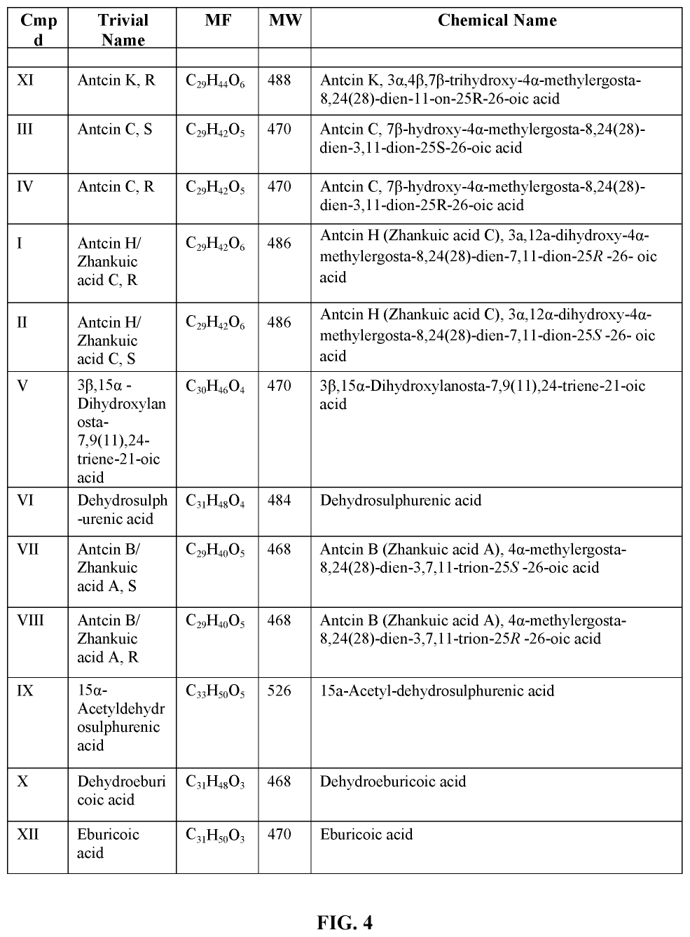

### DETAILED DESCRIPTION OF THE INVENTION

Wild Antrodia cinnamomea (or Antrodia camphorata) is a unique mushroom growing only on Cinnamomum kanehirae in Taiwan. Triterpenoids (I) to (XII) were isolated from the fruit body of Antrodia camphorate. The triterpenoids were examined for their potency for induction of FXR activity using HepG2 FXR-luciferase reporter cells. Out of the twelve triterpenoids, compounds (I) and (II) were found to induce/enhance FXR activity.

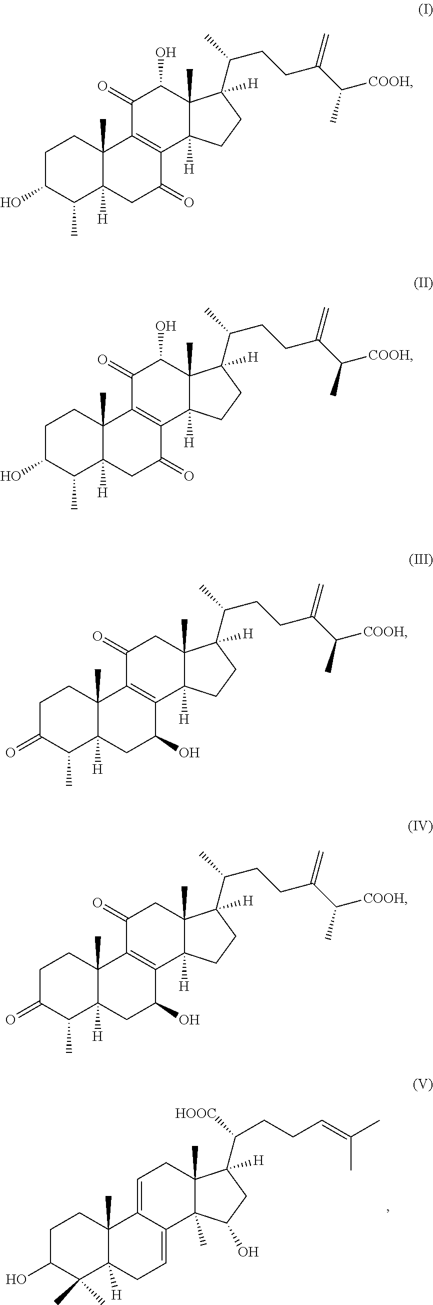

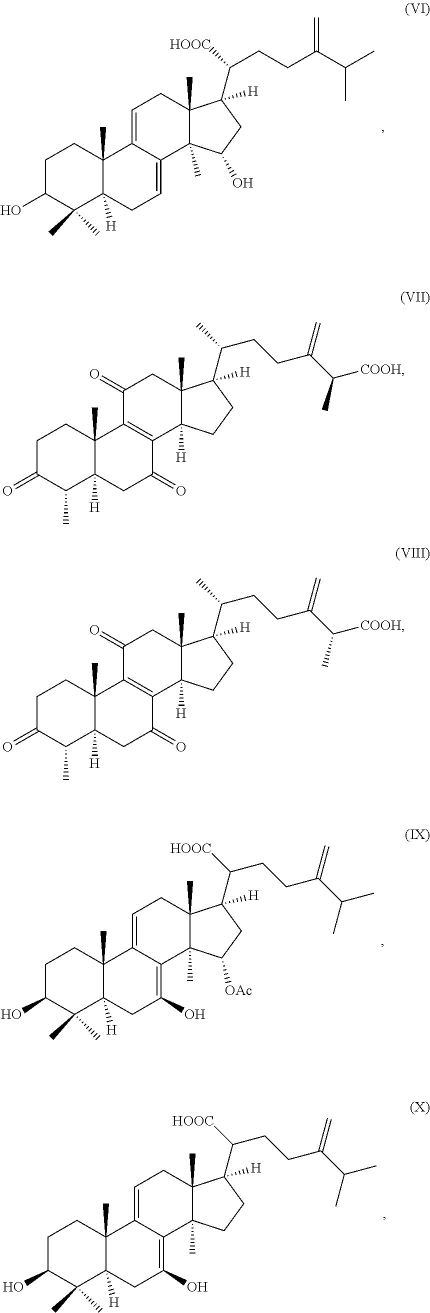

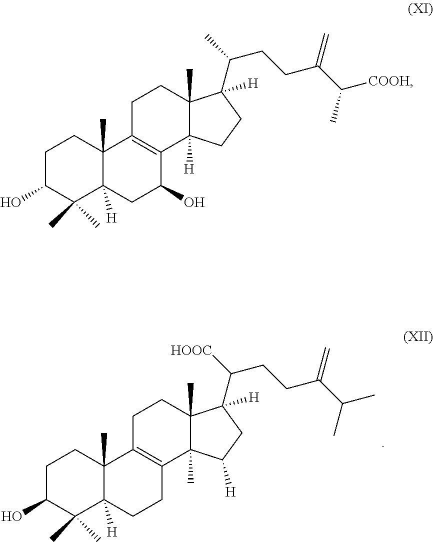

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science and organic chemistry are those well-known and commonly employed in the art. It should be understood that the order of steps or order for performing certain actions is immaterial, so long as the present teachings remain operable. Moreover, two or more steps or actions can be conducted simultaneously or not.

As used herein, the articles “a” and “an” refer to one or to more than one (i.e., to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

As used herein, the term “cancer” is defined as disease characterized by the rapid and uncontrolled growth of aberrant cells. Cancer cells can spread locally or through the bloodstream and lymphatic system to other parts of the body. Examples of various cancers include but are not limited to, bone cancer, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, nasal, pulmonary and topical administration.

A “disease” as used herein is a state of health of an animal wherein the animal cannot maintain homeostasis, and wherein if the disease is not ameliorated then the animal's health continues to deteriorate.

A “disorder” as used herein in an animal is a state of health in which the animal is able to maintain homeostasis, but in which the animal's state of health is less favorable than it would be in the absence of the disorder. Left untreated, a disorder does not necessarily cause a further decrease in the animal's state of health.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or downregulate a protein, a gene, and an mRNA stability, expression, function and activity, e.g., antagonists.

The terms “patient,” “subject” or “individual” are used interchangeably herein, and refer to any animal, or cells thereof whether in vitro or in situ, amenable to the methods described herein. In a non-limiting embodiment, the patient, subject or individual is a human. In other embodiments, the patient is a non-human mammal including, for example, livestock and pets, such as ovine, bovine, porcine, canine, feline, and murine mammals. In yet other embodiments, the patient is an avian animal or bird. Preferably, the patient, individual or subject is human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

The term “prevent,” “preventing” or “prevention,” as used herein, means avoiding or delaying the onset of symptoms associated with a disease or condition in a subject that has not developed such symptoms at the time the administering of an agent or compound commences.

A “therapeutic” treatment is a treatment administered to a subject who exhibits signs of pathology, for the purpose of diminishing or eliminating those signs.

As used herein, the term “therapeutically effective amount” refers to an amount that is sufficient or effective to prevent or treat (delay or prevent the onset of, prevent the progression of, inhibit, decrease or reverse) a disease or condition described or contemplated herein, including alleviating symptoms of such disease or condition.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual and partial numbers within that range, for example, 1, 2, 2.7, 3, 4, 5, 5.3, and 6. This applies regardless of the breadth of the range.

The following abbreviations are used herein: FXR=Farnesoid X Receptor; RXR=Retinoid X receptor; LXR=liver X receptor; CDCA=chenodeoxycholic acid; CYP7A1=cholesterol 7 alpha-hydroxylase; PPARa=peroxisome proliferator-activated receptor alpha; BACS=bile acid-CoA synthetase.

CDCA has the structure:

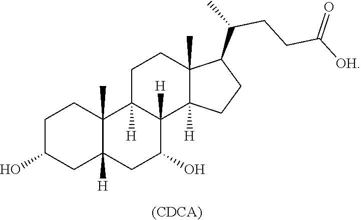

### Methods

In certain embodiments, the invention provides a method of treating at least one disease or disorder in a subject in need thereof. In certain embodiments, the method comprises administering to the subject a therapeutically effective amount of an extract comprising at least one Farnesoid X receptor (FXR) agonist obtained from Antrodia cinnamomea (Antrodia camphorate). In certain embodiments, administering enhances the FXR-mediated transcriptional response in the subject. In certain other embodiments, administering induces FXR-mediated transcriptional response in the subject.

In certain embodiments, the invention provides a method of treating a disease or a disorder related to the intestines, liver, kidney, and/or adrenal glands in a subject in need thereof. In certain embodiments, the method comprises administering to the subject a therapeutically effective amount of a composition comprising at least one FXR agonist obtained from Antrodia cinnamomea or pharmaceutically acceptable salts, solvates, tautomers, or prodrugs thereof. In certain embodiments, the composition is as described elsewhere herein.

In certain embodiments, the invention provides a method of treating at least one disease or disorder in a subject in need thereof, wherein the method comprises administering to the subject a therapeutically effective amount of the composition comprising at least one FXR agonist obtained from Antrodia cinnamomea or pharmaceutically acceptable salts, solvates, tautomers, or prodrugs thereof. In various embodiments, the composition comprises an extract comprising at least one Farnesoid X receptor (FXR) agonist obtained from Antrodia cinnamomea (Antrodia camphorate).

In certain embodiments, the at least one FXR agonist is a triterpenoid compound.

In certain embodiments, the triterpenoid compound includes

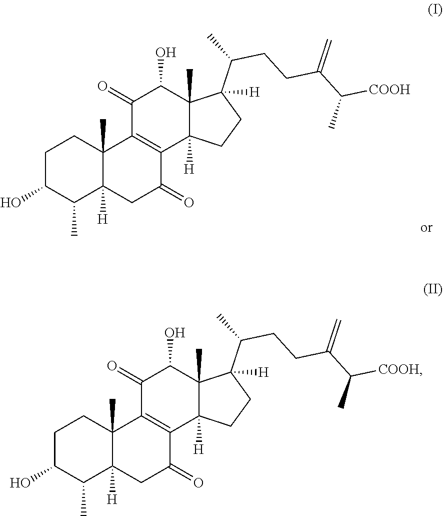 [SMILES: C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@@H](C)C(=O)O ; C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@H](C)C(=O)O]

or salts, solvates, isomers, tautomers, or prodrugs thereof. In some embodiments, a combination of compound (I) and compound (II) can be administered. The relative amounts of compound (I) and compound (II) in the composition can range from 10:1 compound (I):compound (II) to 1:10 compound (I):compound (II).

In certain embodiments, the at least one disease or disorder is related to one selected from intestine, liver, kidney, and adrenal gland. In certain embodiments, the at least one disease or disorder is selected from the group consisting of a liver disease, obesity, diabetes, diarrhea, abdominal pain, hypertension, itchy skin, liver cancer, hepatitis, biliary cholangitis, nonalcoholic steatohepatitis, primary sclerosing cholangitis, inflammation, and fibrosis.

In certain embodiments, administering the composition or extract induces about 60% to about 85% of FXR activity in the absence of chenodeoxycholic acid (CDCA). In certain embodiments, administering induces about 60, 61 62, 63, 64, 65, 66, 67, 68, 69, 70, 71, 72, 73, 74, 75, 76, 77, 78, 79, 80, 81, 82, 83, 84, to about 85% of FXR activity in the absence of chenodeoxycholic acid (CDCA).

In certain embodiments, administering the composition or extract stimulates FXR activity by about 15% to about 30% in the presence of CDCA. In certain embodiments, administering stimulates FXR activity by about 15, 16, 17, 18, 19, 20, 21, 22, 23, 24, 25, 26, 27, 28, 29, to about 30% in the presence of CDCA.

In certain embodiments, administering the composition or extract has no impact on any other hormone receptor signaling pathway. In certain embodiments, administering has no impact on liver X receptor (LXR) mediated transcriptional response. As used herein, the term “no impact” means that administration of any of the compositions or extracts described herein do not increase or decrease a hormone receptor mediated transcriptional response by more than 0.1, 0.5, 1, 2, 3, 4, 5, 6, 7, 8, 9, or 10%. The compositions or extract, in some embodiments, do not increase or decrease a hormone receptor mediated transcriptional response by more than about 0.1 to about 5%, or by more than about 0.1 to about 2%.

In certain embodiments, the FXR agonist is administered in a concentration of about 10 μM to about 85 μM. In certain embodiments, the FXR agonist is administered in a concentration of about 10, 15, 20, 25, 30, 35, 40, 45, 50, 55, 60, 65, 70, 75, 80 or about 85 μM.

In certain embodiments, the administration route is one selected from intravenous, subcutaneous, oral, aerosol, parenteral, ophthalmic, pulmonary, and topical administration.

In certain embodiments, the subject is a mammal. In certain embodiments, the subject is human.

### Compositions

In certain embodiments, the invention provides a pharmaceutical composition comprising at least one FXR agonist selected from:

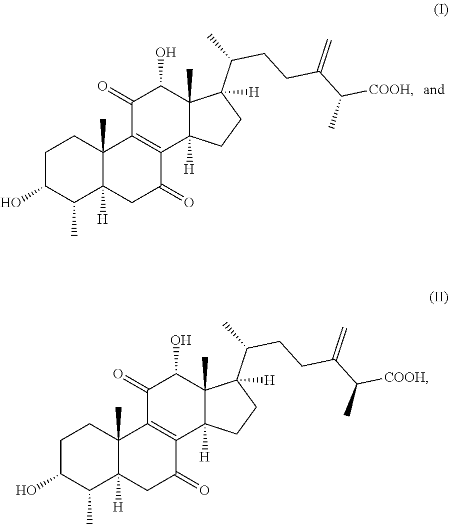 [SMILES: C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@@H](C)C(=O)O ; C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@H](C)C(=O)O]

or pharmaceutically acceptable salts, solvates, tautomers, or prodrugs thereof. In certain embodiments, the composition comprises at least one pharmaceutically acceptable excipient.

In certain embodiments, the composition comprises about 0.0001% to about 0.001% w/w of at least one triterpenoid compound from Antrodia cinnamomea that is neither compound (I) nor compound (II). In certain embodiments, the composition comprises about 0.0001, 0.0002, 0.0003, 0.0004, 0.0005, 0.0006, 0.0007, 0.0008, 0.0009 or about 0.001% w/w of at least one triterpenoid compound from Antrodia cinnamomea that is neither compound (I) nor compound (II)). In certain embodiments, the composition comprises independently about 0.0001, 0.0002, 0.0003, 0.0004, 0.0005, 0.0006, 0.0007, 0.0008, 0.0009 or about 0.001% w/w of at least one of compound (III), compound (IV), compound (V), compound (VI), compound (VII), compound (VIII), compound (IX), compound (X), compound (XI), or compound (XII).

In certain embodiments, the composition comprises about 0.001 to about 0.01% w/w of at least one triterpenoid compound from Antrodia cinnamomea that is neither compound (I) nor compound (II). In certain embodiments, the composition comprises about 0.001, 0.002, 0.003, 0.004, 0.005, 0.006, 0.007, 0.008, 0.009 or about 0.01% w/w of at least one triterpenoid compound from Antrodia cinnamomea that is neither compound (I) nor compound (II). In certain embodiments, the composition comprises independently about 0.001, 0.002, 0.003, 0.004, 0.005, 0.006, 0.007, 0.008, 0.009 or about 0.01% w/w of at least one of compound (III), compound (IV), compound (V), compound (VI), compound (VII), compound (VIII), compound (IX), compound (X), compound (XI), or compound (XII).

In certain embodiments, the composition comprises about 0.01 to 0.1% w/w of at least one triterpenoid compound from Antrodia cinnamomea that is neither compound (I) nor compound (II).

In certain embodiments, the composition comprises about 0.01, 0.02, 0.03, 0.04, 0.05, 0.06, 0.07, 0.08, 0.09 or about 0.1% w/w of at least one triterpenoid compound from Antrodia cinnamomea that is neither compound (I) nor compound (II).

In certain embodiments, the composition comprises about 0.1 to 1% w/w of at least one triterpenoid compound from Antrodia cinnamomea that is neither compound (I) nor compound (II). In certain embodiments, the composition comprises about 0.1, 0.2, 0.3, 0.4, 0.5, 0.6, 0.7, 0.8, 0.9 or about 1% w/w of at least one triterpenoid compound from Antrodia cinnamomea that is that is neither compound (I) nor compound (II). In certain embodiments, the composition comprises independently about 0.1, 0.2, 0.3, 0.4, 0.5, 0.6, 0.7, 0.8, 0.9 or about 1% w/w of at least one of compound (III), compound (IV), compound (V), compound (VI), compound (VII), compound (VIII), compound (IX), compound (X), compound (XI), or compound (XII).

In certain embodiments, the composition comprises a core comprising the at least one FXR agonist coated by the at least one pharmaceutically acceptable excipient or a matrix comprising the at least one FXR agonist interspersed with the at least one pharmaceutically acceptable excipient. In some embodiments, the core is an inert core and the at least one FXR agonist is a coating on the core.

In some embodiments, a prodrug of the compound (I) or compound (II) includes compounds of formula (I-A) or (II-A), respectively:

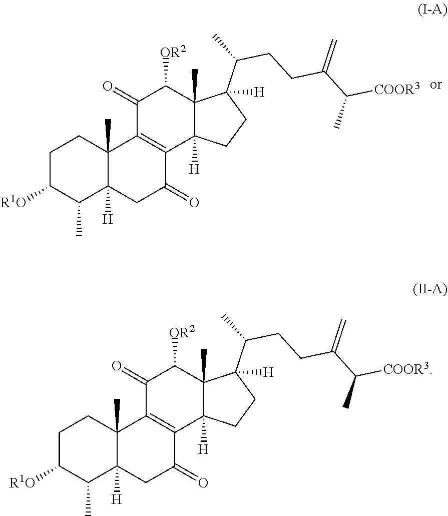

In the compounds of formula (I-A) and (II-A) each of R¹, R², and R³ can be any suitable group that is metabolically or chemically cleaved upon administration of the compound of formula (I-A) or (II-A) in vivo to a subject. In certain embodiments, one or more of R¹, R², and R³ is hydrogen. In other embodiments, at least one of R¹, R², and R³ is not hydrogen.

Non-limiting examples of R¹ and R² include C(═O)—C₁₋₆ alkyl, C(═O)—OC₁₋₆ alkyl, C(═O)-aryl, C(═O)—O-aryl, P(═O)(OH)₂, or an ester of a naturally occurring amino acid. Non-limiting examples of R³ include C₁₋₆ alkyl, aryl, or an ester of a naturally occurring amino acid.

In various embodiments, the compound of formula (I) or formula (I-A) is the only pharmaceutically or therapeutically active agent in the composition. In various embodiments, the compound of formula (II) or formula (II-A) is the only pharmaceutically or therapeutically active agent in the composition. In various embodiments, the compound of formula (I) or formula (I-A) and the compound of formula (II) or formula (II-A) are the only pharmaceutically or therapeutically active agents in the composition.

### Administration/Dosage/Formulations

The regimen of administration may affect what constitutes an effective amount. The therapeutic formulations can be administered to the subject either prior to or after the onset of the disease or the disorder. Further, several divided dosages, as well as staggered dosages can be administered daily or sequentially, or the dose can be continuously infused, or can be a bolus injection. Further, the dosages of the therapeutic formulations can be proportionally increased or decreased as indicated by the exigencies of the therapeutic or prophylactic situation.

Administration of the compositions of the present invention to a patient, preferably a mammal, more preferably a human, can be carried out using known procedures, at dosages and for periods of time effective to treat or ameliorate, or prevent disease or disorder. An effective amount of the therapeutic compound necessary to achieve a therapeutic effect may vary according to factors such as the state of the disease or disorder in the patient; the age, sex, and weight of the patient; and the ability of the therapeutic compound to treat, ameliorate, or prevent disease or disorder. Dosage regimens can be adjusted to provide the optimum therapeutic response. For example, several divided doses can be administered daily or the dose can be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention can be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle.

The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of heart failure in a patient.

In certain embodiments, the compositions of the invention are formulated using one or more pharmaceutically acceptable excipients or carriers. In certain embodiments, the pharmaceutical compositions of the invention comprise a therapeutically effective amount of a compound of the invention and a pharmaceutically acceptable carrier.

The carrier can be a solvent or dispersion medium containing, for example, water, ethanol, polyol (for example, glycerol, propylene glycol, and liquid polyethylene glycol, and the like), suitable mixtures thereof, and vegetable oils.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration can be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of the disease or disorder.

Formulations can be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations can be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, buccal, sublingual or topical. The compounds for use in the invention can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

Oral Administration

For oral application, particularly suitable are tablets, dragees, liquids, drops, suppositories, or capsules, caplets and gelcaps. The compositions intended for oral use can be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets can be uncoated or they can be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

Parenteral Administration

For parenteral administration, the compounds of the invention can be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

Controlled Release Formulations and Drug Delivery Systems

In certain embodiments, the formulations of the present invention can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time can be as long as a month or more and should be a release which is longer that the same amount of agent administered in bolus form.

For sustained release, the compounds can be formulated with a suitable polymer or hydrophobic material which provides sustained release properties to the compounds. As such, the compounds for use the method of the invention can be administered in the form of microparticles, for example, by injection or in the form of wafers or discs by implantation.

In certain embodiments of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

Dosing

The therapeutically effective amount or dose of a compound of the present invention depends on the age, sex, and weight of the patient, the current medical condition of the patient and the progression of heart failure in the patient being treated. The skilled artisan is able to determine appropriate dosages depending on these and other factors.

A suitable dose of a compound of the present invention can be in the range of from about 0.01 mg to about 5,000 mg per day, such as from about 0.1 mg to about 1,000 mg, for example, from about 1 mg to about 500 mg, such as about 5 mg to about 250 mg per day. The dose can be administered in a single dosage or in multiple dosages, for example from 1 to 4 or more times per day. When multiple dosages are used, the amount of each dosage can be the same or different. For example, a dose of 1 mg per day can be administered as two 0.5 mg doses, with about a 12-hour interval between doses.

It is understood that the amount of compound dosed per day can be administered, in non-limiting examples, every day, every other day, every 2 days, every 3 days, every 4 days, or every 5 days. For example, with every other day administration, a 5 mg per day dose can be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the invention can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

The disclosures of each and every patent, patent application, and publication cited herein are hereby incorporated herein by reference in their entirety. While this invention has been disclosed with reference to specific embodiments, it is apparent that other embodiments and variations of this invention can be devised by others skilled in the art without departing from the true spirit and scope of the invention. The appended claims are intended to be construed to include all such embodiments and equivalent variations.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Materials and Methods:

FXR luciferase reporter cells—FXR luciferase reporter cells—HepG2 cancer cells were used in the screening study. HepG2 cells were stably transfected with either FXR reporter DNA in which 3 repeated response element (TTCCCAGGGTCATTGTCCTCTGATG)(SEQ ID NO:2) was inserted in-PGL4.2 luciferase reporter plasmid or LXR reporter DNA in which 3 repeated response element (GGCAAGAGGTAACTGTCGGTCAAATCCT)(SEQ ID NO:3) was inserted in PGL4.2 luciferase reporter plasmid. Reporter cells were selected by puromycin (1 ug/mL) in RPMI-1640 medium with 10% FBS.

Luciferase assay—Reporter cells were treated with compounds at 0, 10, 20, 40, and 80 for 24h in a 37° C.-CO₂ incubator with or without chenodeoxycholic acid (CDCA) for FXR reporter cells or GW3965 for LXR reporter cells. Cells were lysed using luciferase lysis buffer after which luciferase buffer with luciferin was added to generate luminescence. Luminescence was recorded using a luminescence microplate reader.

Real Time Quantitative PCR (RT-qPCR)—Real Time Quantitative PCR (RT-qPCR) for FXR target genes: RNA was extracted from NZZ compounds treated cells using the Roche High Pure RNA isolation kit. cDNA was then generated from RNA samples using Bio-rad iScript Advanced cDNA synthesis kit for RT-qPCR. qPCR was performed using primers as shown in the Table 1 and iTaq™ Universal SYBR® Green Supermix in CFX PCR machine (Bio-rad). Relative mRNA expression was calculated based on the change of the threshold cycle relative to the internal control, (3-actin, using a standard curve generated by purified PCR products.

TABLE 1
qRT-PCR primer for FXR target genes.

| SEQ | | | |
|---|---|---|---|
| ID | | | |
| NO: | Gene name | Primer name | Primer sequence |
| 4 | Cytochrome P450 Family 7 | CYP7A1-F1 | TTGCTACTTCTGCGAAGGCATTTG |
| | Subfamily A Member 1 | | |
| | (CYP7A1) | | |
| 5 | | CYP7A1-R1 | AGGAGACTGGAGGTCTCATGATAC |
| 6 | bile acid-CoA synthetase | BACS-F2 | CTCGGGGACACCTTCCGATGGAAG |
| | (BACS) | | |
| 7 | | BACS-R2 | CACCTTACCCTCACAACCTGGCAC |
| 8 | Kruppel Like Factor 11 | KLF11-F1 | TCTTTATCGACTCTGTGCATAAC |
| | (KLF11) | | |
| 9 | | KLF11-R1 | CTCAGAGCTCTGGCCACTACG |
| 10 | Peroxisome proliferator- | PPARA-F1 | GTGTGAAGGCTGCAAGGGCTTC |
| | activated receptor alpha | | |
| | (PPARa) | | |
| 11 | | PPARA-R1 | TCGTCCAAAACGAATCGCGTTG |
| 12 | β-actin (Actin) | Actin-F | GCCACGGCTGCTTCCAGCTCC |
| 13 | | Actin-R | TTGTGCTGGGTGCCAGGGCAGTGA |

### Example 1: Inhibition of Cholesterol 7 Alpha-Hydroxylase (CYP7A1)

For inhibition of Cholesterol 7 alpha-hydroxylase (CYP7A1) by triterpenoid, it was observed that the inhibition of CYP7A1 by compound (I), at a concentration of about 35 μM to 45 is about 40% stronger than the inhibition of CYP7A1 by compound (II) or by CDCA.

### Example 2: Peroxisome Proliferator-Activated Receptor Alpha (PPARa) mRNA Expression

It was observed that the compound (I), at a concentration of about 75 μM to 85 induces about 2 fold higher PPARa mRNA expression than that induced by compound (II).

### Example 3: Bile Acid-CoA Synthetase (BACS) mRNA Expression

It was observed that the BACS mRNA expression induced by compound (I) or compound (II) is about 2 fold lower than the CDCA induced BACS expression.

### Example 4: KLF11 mRNA Expression

It was observed that compound (I) or compound (II), at a concentration of about 75 μM to 85 induces about 3 to about 5 fold higher KLF11 mRNA expression than that in the absence of the triterpenoid and/or CDCA.

### Example 5: Effects of Triterpenoids on Hormone Signaling Pathways

Table 2 shows selectivity of twelve pure compounds for different hormone receptor signaling pathways.

TABLE 2

| | FXR | | LXR | | MR | | ER-α | | ER-β | | VDRE | | AR | | GRE | |
|---|---|---|---|---|---|---|---|---|---|---|---|---|---|---|---|---|
| compund\ligand | | CDCA | | GW3965 | | Aldosterone | | E2 | | E2 | | VD3 | | DHT | | Dexa |
| XI | — | — | — | — | — | — | — | — | — | — | — | — | — | — | — | — |
| III | — | — | — | — | — | — | — | — | — | — | — | — | — | — | — | — |
| IV | — | — | — | — | — | — | — | ↓70 uM | — | — | — | — | — | — | — | — |
| I | ↑25 uM | — | — | — | — | — | — | — | — | — | — | — | — | — | — | — |
| II | ↑40 uM | — | — | — | — | — | — | — | — | — | — | — | — | — | — | — |
| V | — | — | — | — | — | — | — | — | — | — | — | — | — | — | — | — |
| VI | — | — | — | — | — | — | — | — | — | — | — | — | — | — | — | — |
| VII | — | — | — | — | — | — | — | — | — | ↓40 uM | — | ↓20 uM | — | ↓40 uM | — | — |
| VIII | — | — | — | — | — | — | — | — | — | — | — | ↓25 uM | — | ↓25 uM | — | — |
| IX | — | — | — | — | — | — | — | — | — | — | — | — | — | — | — | — |
| X | — | — | — | — | — | — | — | — | — | — | ↑1.5 uM | — | — | — | — | — |
| XII | — | — | — | — | — | — | — | — | — | — | ↑1.5 uM | — | — | — | — | — |

Within table:
↑ = stimulation following EC₅₀ value.
↓ = inhibition following IC₅₀ value.

Among the twelve purified compounds, compounds (I) and (II) act as agonists to induce FXR mediated transcriptional response. Compounds (I) and (II) had no impact on other hormone receptor signaling pathways.

Compound (IV) could inhibit E2 triggered ERα mediated transcriptional response with IC₅₀ about 80 μM.

Compound (VII) could inhibit E2, VD3, DHT action on ERβ (IC₅₀=40 μM), VDR (IC₅₀=20 μM) and AR (IC₅₀=40 μM).

Compound VIII could inhibit VD3, DHT action on VDR (IC₅₀=25 μM) and AR (IC₅₀=25 μM). Compound X and XII could stimulate VDR activity in a biphasic model of action (EC₅₀=1.5 μM).

### ENUMERATED EMBODIMENTS

The following exemplary embodiments are provided, the numbering of which is not to be construed as designating levels of importance:

Embodiment 1 provides a method of treating at least one disease or disorder in a subject in need thereof, the method comprising:

administering to the subject a therapeutically effective amount of an extract comprising at least one selective Farnesoid X receptor (FXR) agonist obtained from Antrodia cinnamomea (Antrodia camphorate), and

enhancing or inducing FXR mediated transcriptional response in the subject.

Embodiment 2 provides the method of embodiment 1, wherein the at least one FXR agonist is a triterpenoid compound.

Embodiment 3 provides the method of any one of embodiments 1-2, wherein the triterpenoid compound is:

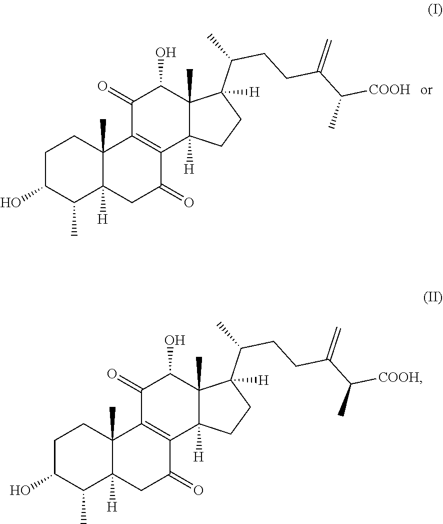 [SMILES: C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@@H](C)C(=O)O ; C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@H](C)C(=O)O]

and salts, solvates, isomers, tautomers, or prodrugs thereof.

Embodiment 4 provides the method of any one of embodiments 1-3, wherein the at least one disease or disorder is related to one selected from intestine, liver, kidney, and adrenal gland.

Embodiment 5 provides the method of any one of embodiments 1-4, wherein the at least one disease or disorder is selected from the group consisting of a liver disease, obesity, diabetes, diarrhea, abdominal pain, hypertension, itchy skin, liver cancer, hepatitis, biliary cholangitis, nonalcoholic steatohepatitis, primary sclerosing cholangitis, inflammation, and fibrosis.

Embodiment 6 provides the method of any one of embodiments 1-5, wherein the administering induces about 60% to about 85% of FXR activity in the absence of chenodeoxycholic acid (CDCA).

Embodiment 7 provides the method of any one of embodiments 1-6, wherein the administering stimulates FXR activity by about 15% to about 30% in the presence of CDCA.

Embodiment 8 provides the method of any one of embodiments 1-7, wherein the administering has no impact on any other hormone receptor signaling pathway.

Embodiment 9 provides the method of any one of embodiments 1-8, wherein the administering has no impact on liver X receptor (LXR) mediated transcriptional response.

Embodiment 10 provides the method of any one of embodiments 1-9, wherein the concentration of about 35 μM to 85 μM.

Embodiment 11 provides the method of any one of embodiments 1-10, wherein the subject is a mammal.

Embodiment 12 provides the method of any one of embodiments 1-11, wherein the subject is human.

Embodiment 13 provides a method of treating an intestinal, liver, kidney, or adrenal gland disease or disorder in a subject in need thereof, the method comprising administering to the subject a therapeutically effective amount of a composition comprising at least one FXR agonist selected from the group consisting of

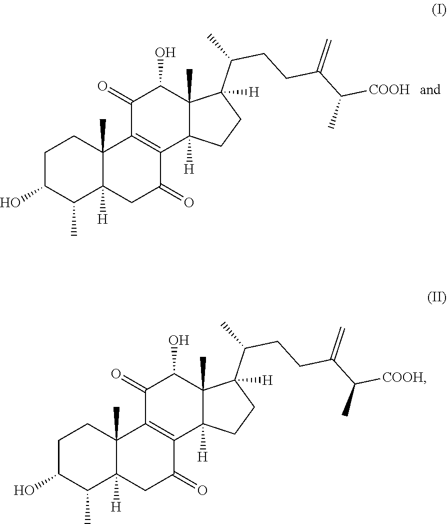 [SMILES: C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@@H](C)C(=O)O ; C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@H](C)C(=O)O]

or pharmaceutically acceptable salts, solvates, tautomers, or prodrugs thereof.

Embodiment 14 provides the method of embodiments 1-13, wherein the administering is by a route selected from the group consisting of intravenous, subcutaneous, oral, aerosol, parenteral, ophthalmic, pulmonary, and topical administration.

Embodiment 15 provides a pharmaceutical composition comprising at least one FXR agonist selected from the group consisting of

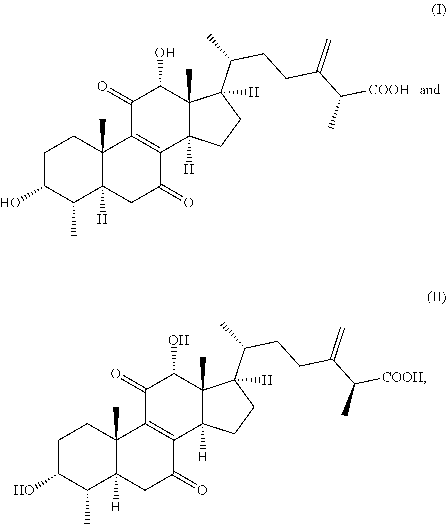 [SMILES: C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@@H](C)C(=O)O ; C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@H](C)C(=O)O]

or pharmaceutically acceptable salts, solvates, tautomers, or prodrugs thereof; and at least one pharmaceutically acceptable excipient, wherein the composition comprises about 0.0001% to about 1% w/w of at least one triterpenoid compound from Antrodia cinnamomea that is neither compound (I) nor compound (II).

Embodiment 16 provides the pharmaceutical composition of embodiment 15, wherein the composition comprises a core comprising the at least one FXR agonist coated by the at least one pharmaceutically acceptable excipient or a matrix comprising the at least one FXR agonist interspersed with the at least one pharmaceutically acceptable excipient.

Embodiment 17 provides a method of treating at least one disease or disorder in a subject in need thereof, the method comprising administering to the subject a therapeutically effective amount of the composition of embodiments 15-16.

Embodiment 18 provides the method of embodiment 17, wherein the disease or disorder is selected from the group consisting of a liver disease, obesity, diabetes, diarrhea, abdominal pain, hypertension, itchy skin, liver cancer, hepatitis, biliary cholangitis, nonalcoholic steatohepatitis, primary sclerosing cholangitis, inflammation, and fibrosis.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating at least one disease or disorder in a subject in need thereof, the method comprising:
administering to the subject a therapeutically effective amount of an extract comprising at least one selective Farnesoid X receptor (FXR) agonist obtained from Antrodia cinnamomea (Antrodia camphorate), and
enhancing or inducing FXR mediated transcriptional response in the subject.

2. The method of claim 1, wherein the at least one FXR agonist is a triterpenoid compound.

3. The method of claim 2, wherein the triterpenoid compound is: 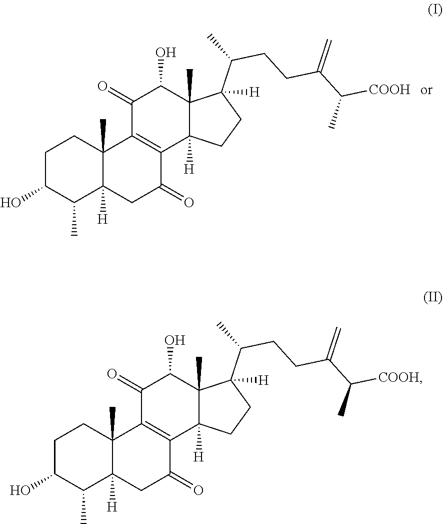 [SMILES: C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@@H](C)C(=O)O ; C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@H](C)C(=O)O]
or salts, solvates, isomers, tautomers, or prodrugs thereof.

4. The method of claim 1, wherein the at least one disease or disorder is related to one selected from intestine, liver, kidney, and adrenal gland.

5. The method of claim 1, wherein the at least one disease or disorder is selected from a liver disease, obesity, diabetes, diarrhea, abdominal pain, hypertension, itchy skin, liver cancer, hepatitis, biliary cholangitis, nonalcoholic steatohepatitis, primary sclerosing cholangitis, inflammation, and fibrosis.

6. The method of claim 1, wherein the administering induces about 60% to about 85% of FXR activity in the absence of chenodeoxycholic acid (CDCA).

7. The method of claim 1, wherein the administering stimulates FXR activity by about 15% to about 30% in the presence of CDCA.

8. The method of claim 1, wherein the administering has no impact or insignificant impact on any other hormone receptor signaling pathway in the subject.

9. The method of claim 1, wherein the administering has no impact or insignificant impact on liver X receptor (LXR) mediated transcriptional response.

10. The method of claim 1, wherein the concentration of the triterpenoid is about 10 μM to 85 μM.

11. The method of claim 1, wherein the subject is a mammal.

12. The method of claim 11, wherein the subject is human.

13. A method of treating an intestinal, liver, kidney, or adrenal gland disease or disorder in a subject in need thereof, the method comprising:
administering to the subject a therapeutically effective amount of a composition comprising at least one FXR agonist selected from the group consisting of 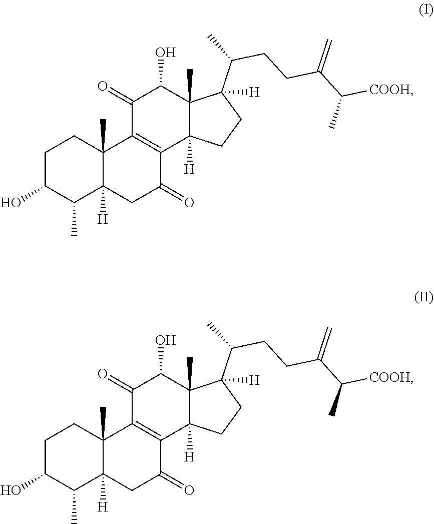 [SMILES: C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@@H](C)C(=O)O ; C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@H](C)C(=O)O]
and
pharmaceutically acceptable salts, solvates, tautomers, or prodrugs thereof.

14. The method of claim 13, wherein the administering is by a route selected from the group consisting of intravenous, subcutaneous, oral, aerosol, parenteral, ophthalmic, pulmonary, and topical administration.

15. A pharmaceutical composition comprising at least one FXR agonist selected from the group consisting of 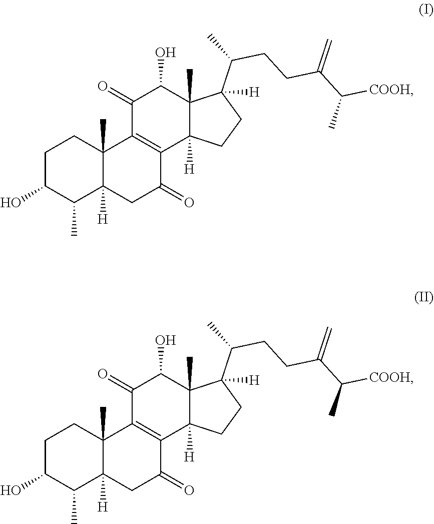 [SMILES: C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@@H](C)C(=O)O ; C=C(CC[C@@H](C)[C@H]1CC[C@H]2C3=C(C(=O)[C@H](O)[C@]12C)[C@@]1(C)CC[C@@H](O)[C@@H](C)[C@@H]1CC3=O)[C@H](C)C(=O)O]
and pharmaceutically acceptable salts, solvates, tautomers, or prodrugs thereof; and
at least one pharmaceutically acceptable excipient,
wherein the composition comprises about 0.0001% to about 1% w/w of at least one triterpenoid compound from Antrodia cinnamomea that is not (I) or (II).

16. The pharmaceutical composition of claim 15, wherein the composition comprises a core comprising the at least one FXR agonist coated by the at least one pharmaceutically acceptable excipient or a matrix comprising the at least one FXR agonist interspersed with the at least one pharmaceutically acceptable excipient.

17. A method of treating at least one disease or disorder in a subject in need thereof, the method comprising administering to the subject a therapeutically effective amount of the composition of claim 15.

18. The method of claim 17, wherein the disease or disorder is selected from a liver disease, obesity, diabetes, diarrhea, abdominal pain, hypertension, itchy skin, liver cancer, hepatitis, biliary cholangitis, nonalcoholic steatohepatitis, primary sclerosing cholangitis, inflammation, and fibrosis


SEQUENCE LISTING (13 sequences)

SEQ ID NO: 1 - DNA, 13 bp, Artificial Sequence
  FXR-RXR Consensus
  misc_feature (7)..(7): n = any nucleotide
aggtcantgacct

SEQ ID NO: 2 - DNA, 25 bp, Artificial Sequence
  FXR Response Element
ttcccagggtcattgtcctctgatg

SEQ ID NO: 3 - DNA, 28 bp, Artificial Sequence
  LXR Response Element
ggcaagaggtaactgtcggtcaaatcct

SEQ ID NO: 4 - DNA, 24 bp, Artificial Sequence
  CYP7A1-F1
ttgctacttctgcgaaggcatttg

SEQ ID NO: 5 - DNA, 24 bp, Artificial Sequence
  CYP7A1-R1
aggagactggaggtctcatgatac

SEQ ID NO: 6 - DNA, 24 bp, Artificial Sequence
  BACS-F2
ctcggggacaccttccgatggaag

SEQ ID NO: 7 - DNA, 24 bp, Artificial Sequence
  BACS-R2
caccttaccctcacaacctggcac

SEQ ID NO: 8 - DNA, 23 bp, Artificial Sequence
  KLF11-F1
tctttatcgactctgtgcataac

SEQ ID NO: 9 - DNA, 21 bp, Artificial Sequence
  KLF11-R1
ctcagagctctggccactacg

SEQ ID NO: 10 - DNA, 22 bp, Artificial Sequence
  PPARA-F1
gtgtgaaggctgcaagggcttc

SEQ ID NO: 11 - DNA, 22 bp, Artificial Sequence
  PPARA-R1
tcgtccaaaacgaatcgcgttg

SEQ ID NO: 12 - DNA, 21 bp, Artificial Sequence
  Actin-F
gccacggctgcttccagctcc

SEQ ID NO: 13 - DNA, 24 bp, Artificial Sequence
  Actin-R
ttgtgctgggtgccagggcagtga